(19) United States
(12) Patent Application Publication
(10) Pub. No.: US 2007/0099940 A1
(43) Pub. Date: May 3, 2007

(54) Pth agonists

(76) Inventor: Paul Kenneth Spearing, Research Triangle Park, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/597,860

(22) PCT Filed: Feb. 3, 2005
(86) PCT No.: PCT/US05/03247

§ 371 Date: Aug. 10, 2006

Related U.S. Application Data
(60) Provisional application No. 60/543,764, filed on Feb. 11, 2004.

(51) Int. Cl. A61K 31/519; C07D 498/02; C07D 491/02; C07D 487/02

(52) U.S. Cl. 514/260.1; 514/262.1; 544/262; 544/278; 544/255

(57) ABSTRACT

This invention relates to compounds of formula (I) 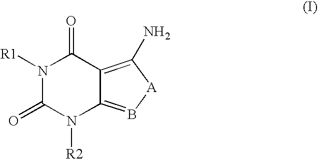 wherein A is S, O, N, or CH; B is S, O, N, or CH;

R¹ and R² are the same or are different and are C₁₋₈ alkyl, C₂₋₈ alkylene, C₃₋₈ cycloalkyl, aryl, heteroaryl, heterocycloalkyl, C₃₋₆ cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵; n is 0, 1 or 2; m is 0, 1 or 2;

R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;

R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or a pharmaceutically acceptable salt or solvate thereof.

### SCOPE OF THE INVENTION

This invention relates to uracil-derived compounds that are agonists of the parathyroid hormone type I receptor (PTH1R) and as such is useful for the treatment of osteoporosis.

### BACKGROUND OF THE INVENTION

Osteoporosis is characterized by bone loss resulting in an increased incidence of fracture. This condition, which is most prevalent in the spine and hip, affects 1 in 3 postmenopausal women, a lesser but significant number of aging men, and is also caused by other conditions including hypogonadism and prolonged glucocorticoid use.

All current therapies to treat osteoporosis, such as bisphosphonates, hormone replacement therapy, SERMs and calcitonin, serve to arrest further bone loss by inhibiting bone resorption (Sato M, et al; 1999, J. Med. Chem. 42:1-24). However, although continued bone loss may be slowed or even prevented by these treatments, new bone formation leading to increased bone mass and strength, does not occur. Consequently, there is a considerable demand for a therapeutic agent capable of stimulating bone formation and that could be used either alone or in combination with an anti-resorptive agent to reduce further risk of fracture. Such a therapeutic agent would be beneficial both to patients who are at risk of developing osteoporosis or who present with established osteoporosis.

Parathyroid hormone (PTH) is a major regulator of calcium homeostasis and acts, in part, by mobilizing calcium from the skeleton through increased bone resorption. Additionally, pulsatile administration of PTH has repeatedly been demonstrated to stimulate new bone formation, both in laboratory animals and in humans (Hock JM, Gera I. 1992. J. Bone Miner. Res. 7:65-72; Wronsld TJ, et al, 1993, Endocrinology 132:823-831; and Reeve J, et al, 1980, Br Med J. 280:134-1344). As such, it is the only agent known to stimulate bone formation on previously quiescent bone surfaces (Hodsman AB, et al, Bone 14:523-527 and Dobnig H, Turner RT. 1995, Endocrinology 136:3632-3638). Indeed, hPTH(1-34), an N-terminal fragment of human PTH that appears to exhibit equivalent bone anabolic activity to the full-length hormone [PTH(1-84)], has been developed by Eli Lilly for the treatment of osteoporosis (Forteo/Teriparatide), as has recombinant human PTH(1-84) by Allelix (Ashworth LE, 2002, Formulary 37:129-139). A recombinant human parathyroid hormone fragment with anabolic actions for treatment of osteoporosis. Formulary 37:129-139) In a clinical trial, PTH(1-34) administered by daily subcutaneous injection for up to 2 years to postmenopausal women with prior vertebral fractures, was reported to reduce fracture incidence at the spine and non-vertebral sites by 65 and 40%, respectively (Neer RM, et al, N. Engl. J. Med. 344:1434-1441).

Taken together, there is overwhelming evidence to suggest that targeting of the receptor for PTH with a small molecule agonist mimicking the actions of PTH(1-34), would be a suitable approach for generating an anabolic response in bone.

PTH elicits its effects by binding and activating a class B G protein-coupled receptor of the 7 transmembrane superfamily, designated PTH1R (Abou-Samra A-B, et al, Proc. Natl. Acad. Sci. USA 89:2732-2736). The PTH1R activates multiple signaling pathways, but predominantly the adenylyl cyclase/cyclic AMP and the phospholipase C/calcium mobilization pathways. Evidence from the literature suggests that activation of the cAMP pathway is necessary but not sufficient for the bone anabolic response (Hock JM, et al, Endocrinology 125:2022-2027 and Rixon RH, et al, J Bone Miner. Res. 9:1179-1189). Both these responses were utilized to identify PTH1R activators (agonists) in screening compounds for agonist activity.

The goal of this invention is to provide a small molecule that mimicks the desired bone anabolic effects of PTH through targeting of the PTH1R, but which can be administered orally rather than by injection. This would offer significant benefits both in terms of lower production costs versus a peptide as well as ease of administration to the patient. Such compounds are provided herein.

### SUMMARY OF THE INVENTION

In one aspect, this invention relates to compounds of formula (I) 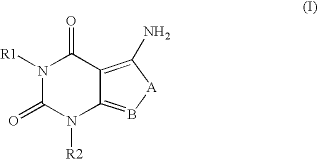
wherein

A is S, O, N, or CH;

B is S, O, N, or CH;

R¹ and R² are the same or are different and are C₁₋₈ alkyl, C₂₋₈ alkylene, C₃₋₈ cycloalkyl, aryl, heteroaryl, heterocycloalkyl, C₃₋₆ cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵;

n is 0, 1 or 2;

m is 0, 1 or 2;

R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;

R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or

a pharmaceutically acceptable salt or solvate thereof.

In another aspect the present invention includes pharmaceutical compositions comprising a compound of formula (I) or a salt or solvate thereof in admixture with a pharmaceutically acceptable excipient, or mixtures thereof.

Another aspect this invention is a means for preventing or treating a condition mediated by PTH which comprises administering to a mammal in need thereof an effective amount of a compound of formula (I), salts or solvates thereof, or mixtures thereof either alone or in admixture with a pharmaceutically excipient.

Another aspect of the invention includes compounds of formula (I) or mixtures thereof for use in the treatment and prevention of diseases and conditions characterised by loss of bone mineral density, mass, or strength, as well as in conditions wherein PTII would have a beneficial pharmacological effect.

Another aspect of the invention includes administering compounds of formula (I) for use as a PTH mimetic.

Another aspect of the invention includes use of the compounds of formula (I) or mixtures thereof in the manufacture of a medicament for use in the treatment of osteopenia and osteoporosis in men and women for reduction in the risk of fractures, both vertebral and nonvertebral.

### DETAILED DESCRIPTION OF THE INVENTION

As used herein, the term “C₁₋₈ alkyl” or “lower alkyl” refers to an alkyl group containing at least 1 and at most 8 carbon atoms. Examples of branched or straight-chain “C₁₋₈ alkyl” groups include, but are not limited to methyl, ethyl, n-propyl, isopropyl, isobutyl, n-butyl, and t-butyl, isobutyl, n-pentyl, n-hexyl, n-heptyl, n-octyl and the like.

The term “alkylene” refers to a straight or branched chain unsaturated aliphatic hydrocarbon radical of 2 to 6 carbon atoms that may be optionally substituted, with multiple degrees of substitution being allowed. Examples of “alkylene” include, but are not limited to methylene, ethylene, n-propylene, n-butylene, and the like.

The term “halogen” refers to fluorine, chlorine, bromine, or iodine.

The term “cycloalkyl” refers to an optionally substituted non-aromatic cyclic hydrocarbon ring of 3 to 8 carbons. Exemplary “cycloalkyl” groups include, but are not limited to cyclopropyl, cyclobutyl, cyclopentyl, cyclohexyl, cycloheptyl and cyclooctyl.

The term “heterocycloalkyl” refers to a heterocyclic ring containing one or more heteroatomic substitutions replacing one or more carbons, selected from S, S(O), S(O)₂, O, or N, that may be further optionally substituted, with multiple degrees of substitution being allowed. Such a ring may be optionally fused to one or more other “heterocycloalkyl” ring(s) or cycloalkyl ring(s). Examples of “heterocyclic” moieties include, but are not limited to tetrahydrofuran, pyran, 1,4-dioxane, 1,3-dioxane, piperidine, pyrrolidine, morpholine, tetrahydrothiopyran, tetrahydrothiophene, and the like.

The term “aryl” refers to a benzene ring or to an optionally substituted benzene ring system fused to one or more optionally substituted benzene rings to form, for example, anthracene, phenanthrene, or naphthalene ring systems. Examples of “aryl” groups include, but are not limited to phenyl, 2-naphthyl, 1-naphthyl, biphenyl, as well as substituted derivatives thereof. The term “lower alkylaryl” further refers to groups of -RₐR_{b}, where Rₐ is a “lower alkyl” as defined herein and Rb is an aryl as defined herein.

“Heteroaryl” refers to a monocyclic aromatic ring system, or to a fused bicyclic aromatic ring system comprising two aromatic rings. These heteroaryl rings contain one or more nitrogen, sulfur, and/or oxygen atoms, where N-oxides and sulfur oxides and dioxides are permissible heteroatom substitutions and may be optionally substituted, with multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. The term “lower alkylheteroaryl” further refers to groups of —RₐR_{b}, where Rₐ is a “lower alkyl” group as defined herein and R_{b} is a heteroaryl as defined herein.

“Alkoxy” refers to the group RₐO—, where Rₐ is alkyl or aryl as defined above. The term “thioalkoxy” refers to the group RₐS—, where Rₐ is alkyl or aryl as defined above. The term “alkoxyaryl” refers to the group R_{b}RₐO—, where Rₐ is alkyl and R_{b} is aryl as defined above.

The terms “C₃₋₆cycloalkylaryl” and “heterocyclylaryl” means a group of —RₐR_{b} where Rₐ is a cycloalkyl or heterocycloalkyl respectively that is fused with R_{b} which is defined as an aryl group. Examples of such groups include:

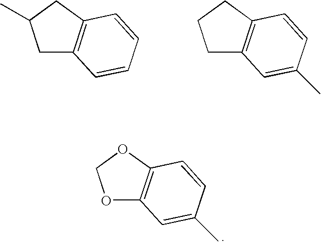

Preferably R¹ and R² independently a C₁₋₆ alkyl, C₃₋₆cycloalkyl, or C₁₋₆ alkylaryl as defined within and A is S and B is N. More preferably R¹ is a C₃₋₆cycloalkyl and R² is an C₁₋₆alkyl as defined herein and A is S and B is N.

Preferred compounds of formula (I) include:

3-amino-5,7-dibutylisothiazolo[3,4-d]pyrimidine-4,6(5H,7H)-dione, and

3 -amino-7-butyl-5-cyclopentylisothiazolo[3 ,4-d]pyrimidine-4,6(5H,7H)-dione.

Certain of the compounds described herein contain one or more chiral atoms, or may otherwise be capable of existing in enantiomeric and diastereomeric forms. The scope of the present invention is intended to cover all isomers per se, as well as mixtures of cis and trans isomers, mixtures of diastereomers, and racemic mixtures of enantiomers. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted.

As noted above, the present invention includes salts and solvates of the compounds of the present invention. Salts include addition salts, metal salts, or optionally alkylated ammonium salts. Examples of such salts include hydrochloric, hydrobromic, hydroiodic, phosphoric, sulfuric, trifluoroacetic, trichloroacetic, oxalic, maleic, pyruvic, malonic, succinic, citric, mandelic, benzoic, cinnamic, methane sulphonic, ethane sulphonic, picric, and the like. Further salts include lithium, sodium, potassium, magnesium, and the like. Reference is also made to Jounal of Pharmaceutical Science, 1997, 66, 2, incorporated herein by reference as relevant to salts.

As used herein, the term “solvate” refers to a complex of variable stoichiometry formed by a solute (in this invention, a compound of formula (I) or a salt or physiologically functional derivative thereof) and a solvent. Such solvents for the purpose of the invention should not interfere with the biological activity of the solute. Examples of solvents include, but are not limited to water, methanol, ethanol, and acetic acid. Preferably the solvent used is a pharmaceutically acceptable solvent. Examples of pharmaceutically acceptable solvents include water, ethanol, and acetic acid.

While it is possible that compounds of the present invention may be administered as the raw chemical, preferably the compounds of the present invention are presented as an active ingredient within a pharmaceutical formulation, as are known in the art. Accordingly, the present invention further includes a pharmaceutical formulation comprising a compound of formula (I), or salt, solvate, or functional derivative thereof together with one or more pharmaceutically acceptable carriers. Optionally, other therapeutic and/or prophylactic ingredients may be included in the pharmaceutical formulation. For example, the compounds of the present invention may be combined with other agents useful in the treatment or prophylaxis of osteoporosis, such as calcium, PTH, Vitamin D, estrogen, SERMs, bisphosphonates, and the like

Formulations of the present invention include those especially formulated for oral, buccal, parental, transdermal, inhalation, intranasal, transmucosal, implant, or rectal administration. Among the variety of administrations, oral administration typically is preferred. For oral administration tablets, capsules, and caplets may contain conventional excipients such as binding agents, fillers, lubricants, disintegrants, and/or wetting agents. Non-limiting examples of binding agents include syrup, acacia, gelatin, sorbitol, tragacanth, mucilage of starch, or polyvinylpyrrolidone (PVP). Non-limiting examples of fillers include, for example, lactose, sugar, microcrystalline cellulose, maize-starch, calcium phosphate or sorbitol. Non-limiting examples of lubricants include, for example, magnesium sterate, stearic acid, talc, polyethylene glycol or silica. Non-limiting examples of disintegrants include, for example, potato starch or sodium starch glycollate. A non-limiting example of a wetting agent includes sodium lauryl sulfate. The tablets additionally may be coated according to methods known in the art.

Alternatively, the compounds of the present invention may be incorporated into oral liquid preparations such as aqueous or oily suspensions, solutions, emulsions, syrups, or elixirs. Moreover, formulations containing these compounds may be presented as a dry product for constitution with water or other suitable vehicle before use. Liquid preparations may contain conventional additives. Non-limiting examples of such additives include suspending agents such as sorbitol syrup, methyl cellulose, glucose/sugar syrup, gelatin, hydroxyethylcellulose, carboxymethyl cellulose, aluminum sterate gel or hydrogenated edible fats. Additionally, emulsifying agents such as lecithin, sorbitan mono-oleate or acacia; non-aqueous vehicles (which may include edible oils) such as almond oil, fractionated coconut oil, oily esters, propylene glycol or ethyl alcohol my be included. Further, preservatives such as methyl or propyl p-hydroxybenzoates or sorbic acid, may be incorporated into the preparation. Such preparations may also be formulated as suppositories, for example, containing conventional suppository bases such as cocoa butter or other glycerides.

Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilising and/or dispersing agents. Alternatively, the active ingredient may be in powder form for constitution with a suitable vehicle, for example, sterile, pyrogen-free water, before use.

The formulations according to the invention may also be formulated as a depot preparation. Such long acting formulations may be administered by implantation, for example, subcutaneously or intramuscularly, or by intramuscular injection. Accordingly, the compounds of the invention may be formulated with suitable polymeric or hydrophobic materials, such as an emulsion in an acceptable oil, ion exchange resins, or as sparingly soluble derivatives, such as a sparingly soluble salt.

Pharmaceutical formulations may be presented in unit dose forms containing a predetermined amount of active ingredient per unit dose. Such a unit may contain certain amounts of a compound of formula (I) depending on the condition being treated, the route of admnistration, and the age, weight and condition of the patient. Preferred unit dosage formulations are those containing a predetermined dose, such as a daily dose, or an appropriate fraction thereof, of an active ingredient. Such pharmaceutical formulations may be prepared by any of the methods well known in the pharmacy art.

A “therapeutically effective amount” of a compound of the present invention will depend upon a number of factors including, for example, the age and weight of the animal, the precise condition requiring treatment and its severity, the nature of the formulation, and the route of administration. Therapeutic effectiveness ultimately will be at the discretion of the attendant physician or veterinarian. An effective amount of a salt or solvate, or physiologically functional derivative thereof, may be determined as a proportion of the effective amount of the compound of formula (I) per se.

No unacceptable toxicological effects are expected when compounds of the present invention are administered in accordance with the present invention.

### METHODS OF PREPARATION—DETAILED DESCRIPTION

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

Preparation of Compounds of Formula I

Compounds of Formula I 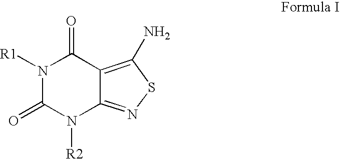
may be prepared from compounds of formula II, below, in a polar, non-protic solvent such as chloroform in the presence of bromine at temperatures of from 0° C. -100° C., such as 20° C.

Compounds of formula II 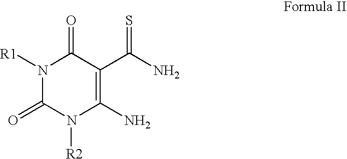
may be prepared from compounds of Formula III 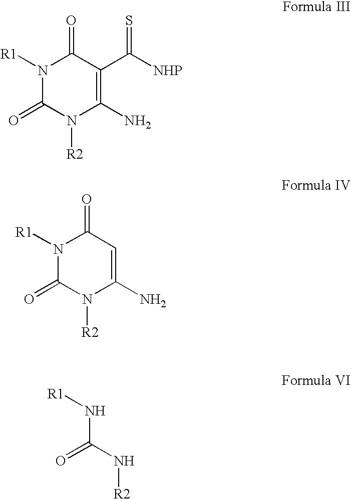
by deprotection of the nitrogen protecting group. Such a protecting group is the 4-methoxy-benzyl protecting group, removed under acidic conditions, such as HBr in acetic acid at temperatures from 20-150° C., such as 80° C. Compounds of formula III may be prepared from compounds of formula IV in a polar aprotic solvent, such as DMF, at temperatures from 20-150° C., such as 100° C. in the presence of a siutable isothiocyanate. The isothiocyanates are commercially available or may be readily prepared by one slilled in the art. Compounds of formula IV may be prepared from compounds of formula VI in a polar aprotic solvent, such as acetic anhydride, and 1 equivalent of cyanoacetic acid at temperatures of from 20° C. to 150° C., such as 80° C. for 2 hr followed by cyclization of the resulting tri-substituted ureas in the presence of a base, such as NaOH, in a polar protic solvent, such as methanol, at temperatures of from 0° C. to 100° C., such as 23° C. Compounds of formula VI are commercially available or may be easily prepared by one skilled in the art (see J. Med. Chem. 1994, 37 (20) 3373-3382).

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

Biological Assay

Compounds of the invention were determined to be agonists of PTH1R using a tagged human PTH1R expressed in CHO cells (Affymax Research, 4001 Miranda Avenue, Palo Alto, Calif. 94304, US) transfected with a cAMP response element (CRE) reporter (EC₅₀=5.3 μM, 73% PTH maximal response). No responses were elicited by compounds of formula (I) in mock-transfected cells, indicating that their stimulatory effects on the cAMP and intracellular calcium ion concentration were PTH1R mediated.

Compounds of formula (I) were found to mimic the effect of PTH(1-34) when added over the concentration range 1-10 μM (EC₅₀ typically ˜1-3 μM), when used in the following assays:

(i) Activation of the cAMP response element-luciferase (CRE-Luc) reporter in HEK cell line expressing human PTH1R (but no response in HEK cells lacking the PTH1R).

(ii) FLIPR/mobilization of intracellular calcium in HEK cells expressing PTH1R.

(iii) Stimulation of cAMP synthesis in the following cells: HEK cells engineered to express the PTH1R; rat osteosarcoma cells (ROS 17/2.8) that express endogenous PTH1R; primary rat osteoblasts isolated from fetal calvariae.

(iv). Stimulation of osteocalcin release from ROS 17/2.8 cells.

(v). Induction of a downstream target gene, RGS-2, in ROS 17/2.8 cells.

Importantly, compounds of formula (I) that were found to be active in the above assays, also caused partial displacement of radio-iodinated (¹²⁵I) (Nle^{8,18})(Tyr³⁴)-PTH(1-34) binding to PTH1R in membrane preparations of HEK cells expressing PTH1R. For these compounds the IC₅₀ value for binding was 2-3 μM, directly coinciding with the concentration range required to observe biological activity.

### EXAMPLES

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### INTERMEDIATES

### Intermediate A

### Preparation of intermediates N-butyl-N-(cyanoacetyl)-N′-cyclopentylurea and N′-butyl-N-(cyanoacetyl)-N-cyclopentylurea:

To 857 μL (8.88 mmol) of cyclopentyl amine in dichloromethane (10 ML) was added 1.0 mL (8.88 mmol) of butylisocyanate (in 5 mL of dichloromethane). The solution was stirred at 23° C. for 15 min then concentrated to give the crude urea which was taken up in acetic anhydride (5 mL) with 790 mg (9.25 mmol) of 2-cyanoacetic acid and heated to 80° C. for 2 hr. The solution was evaporated and the residue taken up in ethyl acetate (50 mL), washed with sat. NaHCO₃ (40 mL of aqueous), water (40 mL) and brine (40 mL) then concentrated and purified by silica gel chromatography eluting with hexanes/ethyl acetate solvent to give 1.22 g (58%) of N-butyl-N-(cyanoacetyl)-N′-cyclopentylurea as a white solid: ¹H NMR (400 MHz, CDCl₃) δ8.80 (bs, 1H), 4.15-4.06 (m, 1H), 3.76 (s, 2H), 3.62 (t, 2H, J=7.6), 2.01-1.93 (m, 2H), 1.74-1.53 (m, 6H ), 1.53-1.41 (m, 2H), 1.41-1.36 (m, 2H), 0.93 (t, 3H, J =7.6); MS (m/z) 252.3 (MH+100%).

Further elution gave 0.48 g (23% yield) of N′-butyl-N-(cyanoacetyl)-N-cyclopentylurea as a white solid: ¹H NMR (400 MHz, CDCl₃) δ6.90 (bs, 1H), 4.31-4.20 (m, 1H), 3.68 (s, 2H), 3.29 (dd, 2H, J_{A}=6.8, J_{B}=13.6), 2.01-1.79 (m, 6H), 1.62-1.50 (m, 4H), 1.43-1.31 (m, 2H), 0.93 (t, 3H, J=7.2); MS (m/z) 252.3 (MH+100%).

### Example 1

### 3-Amino-5-butyl-7-cyclopentylisothiazolo[3,4-d]pyrimidine-4,6(5H,7H)-dione

1(a) Preparation of Intermediate 6-amino-3-butyl-1-cyclopentyl-N-(4-methoxybenzyl)-2,4-dioxo-1,2,3,4-tetrahydropyrimidine-5-carbothioamide:

To 1.22 g of N-butyl-N-(cyanoacetyl)-N′-cyclopentylurea in methanol (10 mL) was added 20% NaOH (5 mL of aqueous) at 0° C. then the solution was stirred at 23° C. for 1 hr. The mixture was concentrated to one half volume, water (20 mL) was added and the solution was extracted twice with ethyl acetate (30 mL). The combined organics were washed with water (30 mL) and brine (30 mL) then dried over NaSO₄ and concentrated to give the crude uracil as a viscous oil. The crude uracil was added to 2.10 g (11.7 mmol) of 1-isothiocyanato-4-methoxybenzene in DMF (15 mL). The solution was stirred at 100° C. for 16 hr. Upon cooling, ethyl acetate (50 mL) was added then the solution was washed three times with water (40 mL), brine (40 mL) and then dried over Na₂SO₄. The solution was concentrated and purified by silica gel chromatography eluting with hexanes/ethyl acetate solvent to give 0.58 g (29% yield) of 6-amino-3-butyl-1-cyclopentyl-N-(4-methoxybenzyl)-2,4-dioxo-1,2,3,4-tetrahydropyrimidine-5-carbothioamide as a colorless oil: ¹H NMR (400 MHz, CDCl₃) δ6.45 (bs, 1H), 5.30 (pent, 1H,J=8.8), 4.03 (t, 2H,J=7.6), 2.16-2.03 (m, 2H), 2.02-1.92 (m, 2H), 1.89-.179 (m, 2H), 1.76-1.66 (m, 2H), 1.66-1.55 (m, 2H), 1.4-1.35 (m, 2H), 0.95 (t, 2H, J =7.2).

1(b) Preparation of 3-amino-5-butyl-7-cyclopentylisothiazolo[3,4-d]pyrimidine-4,6(5H,7H)-dione:

To 310 mg (0.74 mmol) of 6-amino-3-butyl-1-cyclopentyl-N-(4-methoxybenzy)-2,4-dioxo-1,2,3,4-tetrahydropyrimidine-5-carbothioamide was added 30% HBr in acetic acid (10 mL) and the solution heated to 80° C. for 5 hr. The solution was then poured into ice water (25 L) and extracted twice with ethyl acetate (25 mL.) The combined organics were washed with water (25 mL), sat NaHCO₃ (20 mL of aqueous) and brine then dried over Na₂SO₄. The solution was concentrated and purified by silica gel chromatography eluting with hexanes/ethyl acetate solvent to give 102 mg (45% yield) of the thioamide as a white solid. To 40 mg (0.13 mmol) of this thioamide was added bromine (70 μL) in chloroform (1.5 mL) and the solution was stirred at 23° C. for 4 hr. Chloroform (15 mL) was added and the solution was washed with 10% Na₂S₂O₃ (15 mL of aqueous), water (15 mL) and brine (15 mL) then dried over Na₂SO₄, concentrated and purified by silica gel chromatography eluting with hexanes/ethyl acetate solvent to give 25 mg (63% yield) of the title compound as a white solid: ¹H NMR (400 MHz, CDCl₃) δ11.62-11.52 (bs, 1H), 7.24-7.12 (bs, 1H), 5.38-5.28 (m, 1H), 3.93 (t, 2H, J =7.6), 2.10-1.98 (m, 1H), 1.98-1.88 (m, 2H), 1.86-1.76 (m, 2H), 1.76-1.66 (m, 2H), 1.64-1.56 (m, 2H), 1.48-1.39 (m, 2H), 0.99 (t, 3H, j=7.6); MS (m/z) 309.3 (MH+100%).

### Example 2

### 3-Amino-7-butyl-5-cyclopentylisothiazolo[3,4-d]pyrimidine-4,6(5H,7H)-dione

2(a) Preparation of Intermediate 6-amino-1-butyl-3-cyclopentyl-N-(4-methoxybenzyl)-2,4-dioxo- 1,2,3,4-tetrahydropyrimidine-5-carbothioamide:

To 0.48 g of N′-butyl-N-(cyanoacetyl)-N-cyclopentylurea in methanol (5 mL) was added 20% NaOH (3 mL of aqueous) at 0° C. then the solution was stirred at 23° C. for 1 hr. The mixture was concentrated to one half volume, water (20 mL) was added and the solution was extracted twice with ethyl acetate (30 mL). The combined organics were washed with water (30 mL) and brine (30 mL) then dried over NaSO₄ and concentrated to give the crude uracil as a white solid. The crude uracil was added to 710 mg (3.94 mmol) of 1-isothiocyanato-4-methoxybenzene in DMF (5 mL). The solution was stirred at 100° C. for 16 hr. Upon cooling, ethyl acetate (50 mL) was added then the solution was washed three times with water (40 mL), brine (40 mL) and dried over Na₂SO₄. The solution was concentrated and purified by silica gel chromatography eluting with hexanes/ethyl acetate solvent to give 420 mg (57% yield) of 6-arnino-1-butyl-3-cyclopentyl-N-(4-methoxybenzyl)-2,4-dioxo-1,2,3,4-tetrahydropyrimidine-5-carbothioamide as a white solid: ¹H NMR (400 MHz, CDCl₃) δ12.80 (s, 1H), 7.30 (d, 2H, J=8.0), 6.90 (d, 2H, J=8.0), 5.38-5.28 (m, 1H), 4.81 (d, 2H, J=4.8), 3.91 (t, 2H, J =8.0), 3.82 (s, 3H), 2.08-1.99 (m, 2H), 1.99-1.93 (m, 2H), 1.92-1.76 (m, 4H), 1.68-1.58 (m, 2H), 1.54-1.38 (m, 2H), 0.98 (t, 3H, J=7.2).

2(b) Preparation of 3-amino-7-butyl-5-cyclopentylisothiazolo[3,4-d]pyrimidine-4,6(5H,7H)-dione:

To 360 mg (0.86 mmol) of 6-amino-1-butyl-3-cyclopentyl-N-(4-methoxybenzyl)-2,4-dioxo-1,2,3,4-tetrahydropyrimidine-5-carbothioamide was added 30% HBr in acetic acid (10 mL) and the solution heated to 80° C. for 5 hr. The solution was then poured into ice water (25 mL) and extracted twice with ethyl acetate (25 mL.) The combined organics were washed with water (25 mL), sat NaHCO₃ (20 mL of aqueous) and brine then dried over NaSO₄. The solution was concentrated and purified by silica gel chromatography eluting with hexanes/ethyl acetate solvent to give 49 mg (19% yield) of the thioamide as a yellow glass. To 48 mg (0.15 mmol) of this thioamide was added bromine (80 μL) in chloroform (2 mL) and the solution was stirred at 23° C. for 4 hr. Chloroform (15 mL) was added and the solution was washed with 10% Na₂S₂O₃ (15 mL of aqueous), water (15 mL) and brine (15mL) then dried over Na₂SO₄, concentrated and purified by silica gel chromatography eluting with hexanes/ethyl acetate solvent to give 34 mg (72% yield) of the title compound as a white solid: ¹H NMR (400 MHz, CDCl₃) δ6.47 (bs, 1H), 5.32 (pent, 1H, J=8.8), 3.90 (t, 2H, J=7.6), 2.29-2.18 (m, 2H), 2.00-1.86 (m, 4H), 1.66-1.45 (m, 4H), 1.42-1.33 (m, 2H), 0.94 (t, 2H, J =7.6); MS (m/z) 309.3 (MH+100%).

### Example 3

### 3-Amino-5,7-dibutylisothiazolo [3,4-d]pyrimidine-4,6(5H,7H)-dione

Preparation of Intermediate 6-amino-1,3-dibutyl-N-(4-methoxybenzyl)-2,3-dioxo-1,2,3,4-tetrahydropyrimidine-5-carbothioamide: To 445 mg (1.86 mmol) of 6-amino-1,3-dibutyl-1H-pyrimidine-2,4-dione (J. Med. Chem. 1994, 37 (20) 3373-3382) in DMF (5 mL) was added 1.0 g (5.58 mmol) of 1-isothiocyanato-4-methoxybenzene at 23° C. The solution was heated to 100° C. for 16 hr. Upon cooling, ethyl acetate (30 mL) was added and the solution washed with three portions of water (20 mL) and brine (20 mL), dried over Na₂SO₄, concentrated then purfied by silica gel chromatography eluting with hexanes/ethyl acetate solvent to give 540 mg (70%) of 6-amino-1,3-dibutyl-N-(4-methoxybenzyl)-2,3-dioxo- 1,2,3,4-tetrahydropyrimidine-5-carbothioamide as a yellow oil: ¹H NMR (400 MHz, CDCl₃) δ12.79 (s, 1H), 7.28 (d, 2H, J =7.6), 6.87 (d, 2H, J =7.6), 4.80 (d, 2H, J =4.8), 3.99-3.84 (m, 4H), 3.79 (s, 3H), 1.74-1.52 (m, 4H), 1.48-1.31 (m, 4H1), 0.98 (t, 3H, J=7.2), 0.91 (t, 3H, J=7.2); MS (m/z) 419.4 (MH+100%).

To 110 mg (0.26 mmol) of 6-amino-1,3-dibutyl-N-(4-methoxybenzyl)-2,3-dioxo-1,2,3,4-tetrahydropyrimidine-5-carbothioamide in chloroform (2 mL) was added 70 μL (1.32 mmol) of bromine. The solution was stirred at 23° C. for 36 hr. Chloroform (20 mL) was added then the solution was washed with 10% Na₂S₂O₃ (15 mL of aqueous), water (15 mL) and brine (15 mL) then dried over Na₂SO₄, concentrated and purified by silica gel chromatography eluting with hexanes/ethyl acetate solvent to give 24 mg (34% yield) of the title compound as a white solid: ¹H NMR (400 MHz, CDCl₃) δ6.46 (bs, 2H), 4.11 (t, 2H, J=6.8), 3.92 (t, 2H, J=7.6), 1.76-1.59 (m , 4H), 1.44-1.33 (m, 4H), 0.95 (t, 6H, J=7.2); MS (m/z) 297.3 (MH+100%).

What is claimed is:

1. A compound of formula (I) 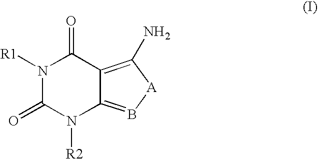
wherein,
A is S, O, N, or CH;
B is S, O, N, or CH;
R¹ and R² are the same or are different and are C₁₋₈ alkyl, C₂₋₈ alkylene, C₃₋₈ cycloalkyl, aryl, heteroaryl, heterocycloalkyl, C₃₋₆ cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O0₂NR⁵R⁶, C(S)NR⁵R⁶, CONR⁵R⁶, C(O)nR⁵;
n is 0, 1 or 2;
m is 0, 1 or 2;
R⁵ is hydrogen, alkyl, aryl, alkylaryl, heterocycloalkyl, or heteroaryl and is unsubstituted or substituted by one or more groups selected from the group consisting of alkyl, C₁₋₈ alkoxy, aryl, heteroaryl, halogen, NO₂, CN, N₃, SCF₃, and CF₃;
R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or
a pharmaceutically acceptable salt or solvate thereof.

2. A compound of claim 1 wherein in formula (I) R¹ and R² are independently a C₁₋₆ alkyl, C₃₋₆ cycloalkyl, or C₁₋₆ alkylaryl as defined within and A is S and B is N.

3. A compound of claim 1 wherein in formula (I) R¹ is a C₃₋₆ cycloalkyl and R² is an C₁₋₆alkyl as defined herein and A is S and B is N.

4. A compound of claim 1 which is 3-amino-5,7-dibutylisothiazolo[3,4-d]pyrimidine-4,6(5H,7H)-dione, and
3-amino-7-butyl-5-cyclopentylisothiazolo[3,4-d]pyrimidine-4,6(5H,7H)-dione; or
a pharmaceutically acceptable salt thereof.

5. A pharmaceutical composition comprising a compound of formula (I) according to claim 1 in admixture with a pharmaceutically acceptable excipient.

6. A method for the prophylaxis of or for treating osteoporosis in a mammal comprising administering a effective amount of a compound of formula (I) according to claim 1 either neat or in admixture with a pharmaceutically acceptable excipient.
